(19) United States
(12) Patent Application Publication
(10) Pub. No.: US 2022/0107491 A1
(43) Pub. Date: Apr. 7, 2022

(54) BORESCOPES WITH ACTIVE OPTICAL LENSES AND RELATED METHODS

(71) Applicant: Xenocor, Inc., Salt Lake City, UT (US)

(72) Inventors: Lane G. Brooks, Highland, UT (US); Richard A. Dixon, Spanish Fork, UT (US); Dennis James Muhlestein, American Fork, UT (US); Anthony J. Laurienti, IV, West Valley, UT (US)

(73) Assignee: Xenocor, Inc., Salt Lake City, UT (US)

(21) Appl. No.: 17/491,493

(22) Filed: Sep. 30, 2021

Related U.S. Application Data
(60) Provisional application No. 63/087,274, filed on Oct. 4, 2020.

(51) Int. Cl. G02B 23/24; G02B 3/14

(52) U.S. Cl. CPC G02B 23/243; G02B 23/2476; G02B 23/2461; G02B 3/14

(57) ABSTRACT

Borescopes, such as chip-on-a-tip laparoscopes and endoscopes, having variable and/or active focus lenses. In some preferred embodiments, a chip-on-a-tip borescope may comprise a tip assembly having a fixed focus lens and an active or variable focus lens. The active lens may be part of an active lens assembly, which may comprise a substrate, such as a printed circuit board, along with an active lens unit that may be configured to receive electrical signals, such as voltage steps, that may be used to change the shape of the lens component of the unit to change the focal distance of the device. The substrate may be physically coupled to other elements of the scope and may be electrically coupled with other elements of the scope, such as a voltage driver that may be provided in a printed circuit board in the handle of the scope, for example.

### CROSS-REFERENCE TO RELATED APPLICATIONS

This application claims the benefit under 35 U.S.C. § 119(e) of U.S. Provisional Patent Application No. 63/087,274, which was filed Oct. 4, 2020 and titled “BORESCOPES WITH ACTIVE OPTICAL LENSES AND RELATED METHODS,” which is hereby incorporated herein by reference in its entirety.

### SUMMARY

Embodiments of apparatus and methods are disclosed herein that relate to borescopes and, in preferred embodiments, medical borescopes, such as laparoscopes, endoscopes, and the like.

Typically, “chip-on-a-tip” laparoscopes and endoscopes comprise a fixed-focus lens, which is set during manufacturing to provide a fixed focal length that is optimal for a particular use. Thus, objects that are either closer to or further from the scope than the focal length will lose focus in proportion to the distance from the set focal length. During use, the operator must therefore move the scope in and out depending on the target object for imaging unless the object is at the preset focal length.

The present inventors have therefore determined that it would be desirable to provide borescopes, and related systems and methods, that overcome one or more of the foregoing limitations and/or other limitations of the prior art. In some embodiments disclosed herein, the inventive concepts disclosed herein may allow for borescopes to adjust the focal distance during use, either manually or automatically, without requiring the user to adjust the physical distance between the tip of the borescope and the object to be imaged.

Some embodiments may therefore comprise a medical borescope having an imaging assembly positioned at or near a distal tip of the scope. The imaging assembly may comprise a fixed focus lens and an active or variable focus lens. In some such embodiments, the active lens may comprise an active lens assembly, which may comprise a substrate, such as a printed circuit board, along with an active lens unit that may be configured to receive electrical signals, such as voltage steps, that may be used to change the shape of the lens component of the unit to change the focal distance of the device. The substrate may be physically coupled to other elements of the scope, such as a tip assembly of the scope, and may be electrically coupled with other elements of the scope as well, such as a voltage driver that may be provided in a printed circuit board in the handle of the scope, for example.

In some embodiments, the active lens unit and/or assembly may comprise a piezoelectric actuator, which may comprise a piezoelectric material that may be configured to reshape under an applied voltage to adjust the focal distance of the device in real time. In some such embodiments, the piezoelectric material may be configured to indirectly refocus the lens, which may, for example, be accomplished by positioning the piezoelectric material adjacent to a thin membrane, such as a glass membrane. The membrane may serve as an actuator to deform a polymer, which may be sandwiched between two transparent layers, such as glass layers. The piezoelectric material may be coupled with a tunable voltage source, such as a voltage driver to allow for real-time focusing of the active lens.

Some embodiments may further comprise polarizers, such as polarizing films, which may be used to reduce undesirable specular highlights during imaging. In some such embodiments, a first polarizing film or other polarizer may be positioned adjacent to the light source used in the scope, such as one or more LEDs in a tip assembly of the scope, to polarize the outgoing light. A second polarizing film or other polarizer may be positioned within the path of the incoming light to polarize the reflected light prior to it hitting the image sensor. Preferably the second polarizer is rotated with respect to the first polarizer, preferably by ninety degrees or about ninety degrees. The second polarizer may be positioned at any point within the incoming light path of the scope, such as adjacent to one of the lenses, camera modules, or adjacent to the image sensor itself.

Some embodiments may comprise, either in addition to or as an alternative to any of the other features/components/elements described herein, a partitioned, flexible printed circuit board defined by a plurality of individual strips, each having a subset of the wires or other electrical coupling elements of the full PCB/assembly. These strips may be interconnected by pieces of the substrate of the PCB, which may or may not include associated wiring, such that the entire assembly and all of its wiring makes up a single unit. The various strips, which may extend parallel, or at least substantially parallel, to one another, may then be folded within a tube of a scope, preferably in an alternating and/or accordion style manner, which may allow the assembly/unit to bend and flex more readily in multiple directions and therefore facilitate use within an articulating borescope tip without causing damage to the flexible PCB, which may otherwise have a single, natural bending direction and may be damaged when bending in other directions.

Still other embodiments may comprise one or more heating elements, such as resistors, that may be configured to reduce condensation on transparent surfaces of the scope exposed to the ambient environment, such as a cover or a lens of a tip assembly of the scope, to more quickly equalize the temperature of such surface(s) with that of a patient's internal body temperature. Preferably, such heating elements are positioned on or near the cover, such as a cover glass, or another external surface.

In a more specific example of a medical borescope according to some embodiments, the borescope may comprise a handle having a tube coupled to the handle, along with a tip assembly positioned at or adjacent to a distal tip of the tube. The tip assembly may comprise a light source; an image sensor; a fixed focus lens; and a variable focus lens configured to allow an operator of the medical borescope to adjust a focal distance of the medical borescope during use.

In some embodiments, the variable focus lens may comprise a variable focus lens assembly comprising a substrate and an active optical lens unit configured to be reshaped during use to change the focal distance of the medical borescope. In some such embodiments, the substrate may comprise a printed circuit board and/or may comprise a central opening configured to allow light to pass therethrough and reach the active optical lens unit.

In some embodiments, the printed circuit board may be configured to provide an electrical interface with the fixed focus lens. The substrate may comprise, in some embodiments, a ring shape having a flat side, which may be configured to allow electrical wires to pass by the substrate within the tube.

Some embodiments may further comprise a tunable voltage source configured to allow for selective adjustment of the focal distance by changing a shape of the variable focus lens, such as a voltage driver.

Some embodiments may comprise a piezoelectric actuator configured to change the focal distance by changing a shape of the variable focus lens.

In some embodiments, the medical borescope may be configured to provide for automatic focusing. Some embodiments may further, or alternatively, be configured to allow a user to manually refocus the variable focus lens during use as desired, such as by way of buttons or other actuators on the handle, for example.

Some embodiments may further comprise a transparent cover, such as a cover glass, which may be positioned at or adjacent to a distal end of the tip assembly. Some such embodiments may comprise one or more heating elements positioned and configured to reduce condensation on the transparent cover during use.

In another specific example of a medical borescope according to some embodiments, the medical borescope may comprise a handle and a shaft, such as a tube, coupled to the handle, wherein at least a portion of the shaft is configured to articulate. An array of LEDs may be positioned at a tip of the shaft. The borescope may further comprise a variable focus lens configured to allow for adjustment of a focal distance of the medical borescope during use, which may be part of a tip assembly. A flexible printed circuit board may extend from the array of LEDs to the handle. The flexible printed circuit board may comprise a plurality of flexible strips, which may extend parallel, or at least substantially parallel, to one another, and may be folded to facilitate bending of the flexible printed circuit board in at least two directions.

Some embodiments may further comprise an image sensor. In some such embodiments, the flexible printed circuit board may be electrically and/or physically coupled with the image sensor.

In some embodiments in which the shaft comprises a tube, the flexible printed circuit board may extend through the tube from the array of LEDs to the handle. In some such embodiments, each of the plurality of flexible strips may be folded within the tube. This folding may preferably be in an alternating or accordion-style manner.

Some embodiments may comprise an image sensor and two polarizers rotated with respect to one another to reduce specular highlights. For example, a first polarized film may be positioned and configured to polarize light from the array of LEDs and a second polarized film may be positioned and configured to polarize light entering the image sensor. In some such embodiments, the first polarized film may be rotated by ninety degrees, or about ninety degrees, with respect to the second polarized film.

In yet another example of a medical borescope according to some embodiments, the borescope may comprise a handle; a tube coupled to the handle; a fixed focus lens; and a variable focus lens assembly. The variable focus lens assembly may comprise a printed circuit board substrate and an active optical lens unit configured to be reshaped during use to change the focal distance of the medical borescope.

In some embodiments, the medical borescope may be configured to provide for either automated or manual refocusing of the variable focus lens assembly.

In some embodiments, the variable focus lens assembly may further comprise a piezoelectric actuator.

The features, structures, steps, or characteristics disclosed herein in connection with one embodiment may be combined in any suitable manner in one or more alternative embodiments.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 depicts a borescope having an active optical lens assembly according to some embodiments;

FIG. 2 is a perspective view of the handle of a borescope having an active optical lens assembly;

FIG. 3 depicts the borescope of FIG. 2 with a portion of the handle shown in phantom;

FIGS. 4A and 4B depict an example of an active optical lens assembly comprising an active lens and a substrate to facilitate coupling of the active optical lens with a borescope;

FIG. 5 depicts a borescope tip assembly comprising an active lens and a fixed lens;

FIG. 6 is an exploded, perspective view of a medical borescope comprising an active optical lens assembly according to other embodiments;

FIG. 7 is a cross-sectional view of the tip assembly of the medical borescope of FIG. 6;

FIG. 8 depicts the distal end of an alternative borescope comprising polarizers according to still other embodiments;

FIGS. 9A-9C are exploded, perspective views depicting three alternative configurations for placement of polarizing films within a borescope;

FIG. 10 is a plan view of a partitioned, flexible PCB assembly comprising interconnected strips according to some embodiments; and

FIGS. 11A-11C are perspective views illustrating how a partitioned, flexible PCB assembly can be folded to fit within a tube of a medical borescope according to some embodiments and implementations.

### DETAILED DESCRIPTION

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure but is merely representative of possible embodiments of the disclosure. In some cases, well-known structures, materials, or operations are not shown or described in detail.

Various embodiments of apparatus and methods are disclosed herein that relate to borescopes and other related medical borescoping, such as laparoscopy, endoscopy, and the like. The present inventors also anticipate possible uses of the inventive teachings provided herein in connection with certain industrial applications.

In some embodiments disclosed herein, medical borescopes are disclosed that comprise active optical elements, such as active lenses using electro-optical components to provide a user with the ability to adjust the focal distance to an object, such as an internal body tissue in the case of a laparoscope or endoscope, either manually or automatically. In some embodiments, the active lens may comprise a piezoelectric material, which may be positioned on adjacent to a thin membrane, such as a glass membrane. The membrane may serve as an actuator to deform a polymer, which may be sandwiched between two transparent layers, such as glass layers. The piezoelectric material may be coupled with a tunable voltage source to allow for real-time focusing of the active lens. When used with a fixed lens as part of a tip and/or focusing assembly of a borescope, the active lens may therefore allow a surgeon or other user to change the focal distance of the assembly to allow for either auto-focusing or manual focusing in real time. An example of an active lens assembly that may be used, or modified for use with, borescopes disclosed herein is the TLens® lens offered by Polight ASA. Examples of suitable optical components, materials, techniques, circuits, assemblies, and the like that may be useable in connection with various embodiments and implementations of the inventions described herein can be found in U.S. Pat. No. 9,964,672 titled METHOD FOR OPTIMIZING A PIEZOELECTRIC ACTUATOR STRUCTURE FOR A DEFORMABLE LENS; U.S. Pat. No. 10,001,629 titled PIEZOELECTRICALLY ACTUATED OPTICAL LENS; U.S. Pat. No. 10,473,900 titled TUNABLE MICROLENS WITH A VARIABLE STRUCTURE ELEMENT; and U.S. Pat. No. 10,720,857 titled ELECTRONIC CIRCUIT FOR CONTROLLING CHARGING OF A PIEZOELECTRIC LOAD, each of which is hereby incorporated by reference in its entirety. As another example of a potentially suitable technology, the VARIOPTIC® lens technology by Corning® may be useful in connection with some embodiments and/or applications.

In some preferred embodiments, the borescope may comprise a handle, a tube, and a tip at the distal end of the tube. The tip may comprise one or more light sources, such as LED lights, one or more image sensors, a lens assembly, which may include both an active and/or tunable lens and a fixed focus lens, and/or other medical borescope components. In some embodiments, the tip may further comprise a PCB and/or a memory element, such as a flash memory component or other non-volatile memory component, which may be used to store various types of data, such as the duration and/or number of uses of the device and/or model identification or calibration data, as described in U.S. Pat. No. 9,943,214 titled MEDICAL BORESCOPES AND RELATED METHODS AND SYSTEMS, which was filed on Dec. 3, 2015 and is also hereby incorporated herein by reference in its entirety.

As also described in the aforementioned patent application incorporated herein by reference, some embodiments may further comprise a dongle and/or interface box, which may be communicatively coupled with the device, such as by way of wires or by being plugged into the device, such as into a port formed within the handle of the device. This dongle/interface box may comprise a memory element and a processor, which may be used to process image data from an image sensor in the device. In some embodiments, the dongle/interface box may be removably coupled with the device so that it can be coupled with a plurality of distinct laparoscopes or other borescopes. For example, the dongle/interface box may comprise a data port that may be used to couple the dongle with a plurality of distinct borescopes and/or other devices, such as a general-purpose computer. In this manner, as discussed above, data obtained from the borescope, such as usage data, may be stored in the memory element of the dongle/interface box and ultimately transferred to another computer/device following a medical procedure.

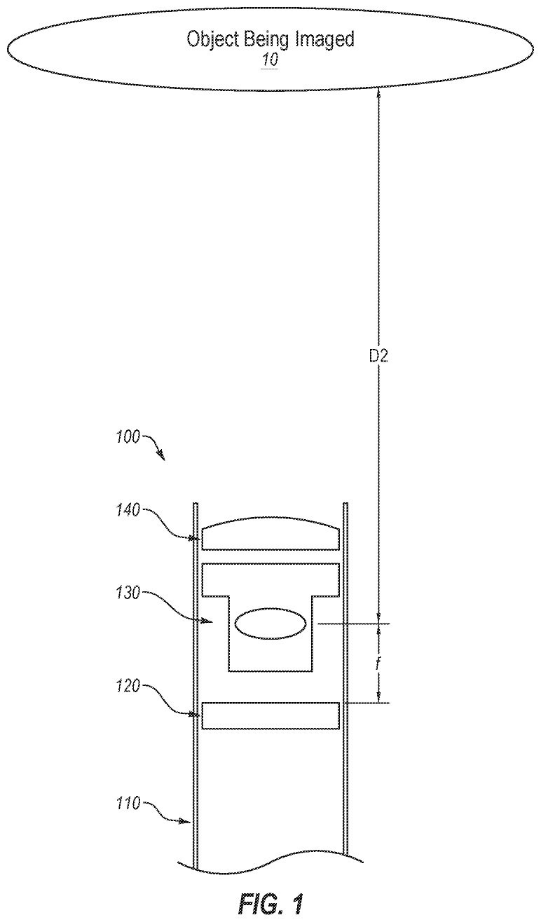

FIG. 1 is a schematic diagram illustrating one example of the tip of a borescope 100 comprising an active and/or tunable optical system, which in the diagram of FIG. 1 is illustrated as including a variable focus lens 140, which may comprise an active and/or tunable lens, and a fixed focus lens 130. An image sensor 120 is positioned behind both the variable focus lens 140 and the fixed focus lens 130 within a shaft or tube 110 of borescope 100.

By including an active/variable focus lens 140, unlike prior art endoscopes, a surgeon may be able to adjust the focal distance to an object being imaged 10, which is represented by distance D2, in real time. Thus, for example, when object 10 comprises an internal body tissue, a surgeon may be able to refocus the image captured on image sensor 120 from another tissue or other object to object 10 without moving the borescope 100 to change the distance D2 so that it matched the distance to the object previously being imaged, as would be the case with prior art laparoscopes and endoscopes. By contrast, as those of ordinary skill in the art will appreciate, typical “chip-on-a-tip” endoscopes set the distance f between the lens 130 and the image sensor 120 during manufacturing and therefore the optical focal distance (D2 in the absence of the variable focus lens 140) cannot be adjusted during use.

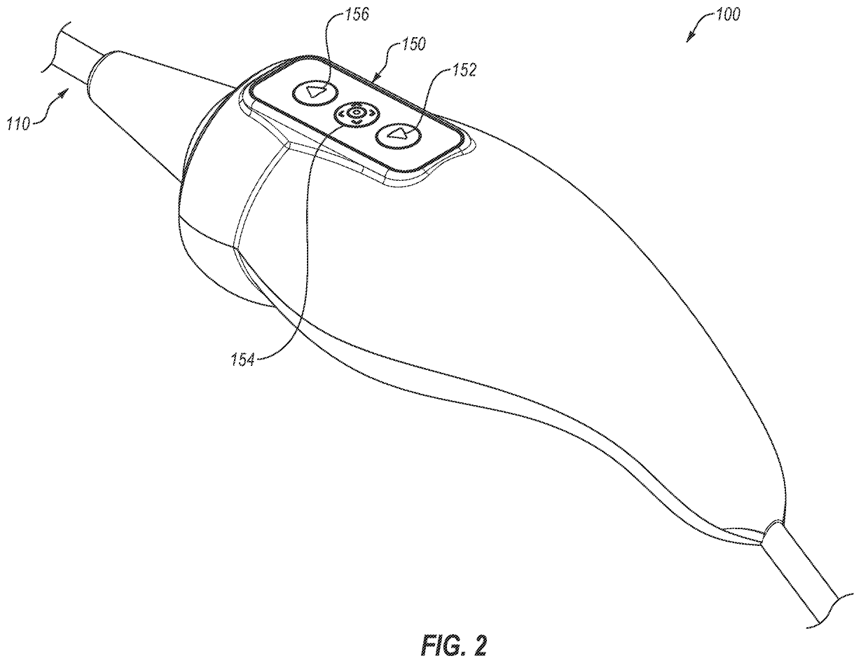

FIG. 2 is a perspective view of a laparoscope 100 illustrating the handle portion of the device. As shown in this drawing, a focusing control panel 150 is provided, which allows a surgeon or other user to control the active/variable focus lens 140. Thus, buttons 152 and 156 may be used to change the focal distance in opposing directions, which may allow a surgeon to precisely focus on a particular tissue or object of interest during a surgical procedure. Some embodiments may also include an auto-focus function, which in the case of the depicted embodiment may be entered by pressing button 154. Of course, as those of ordinary skill in the art will appreciate, some embodiments may be configured to provide for autofocusing capabilities by default and therefore need not provide a button or other actuation means to enter an autofocus mode. Similarly, some embodiments may only provide for manual refocusing and therefore may omit a capability of providing for autofocus, if desired.

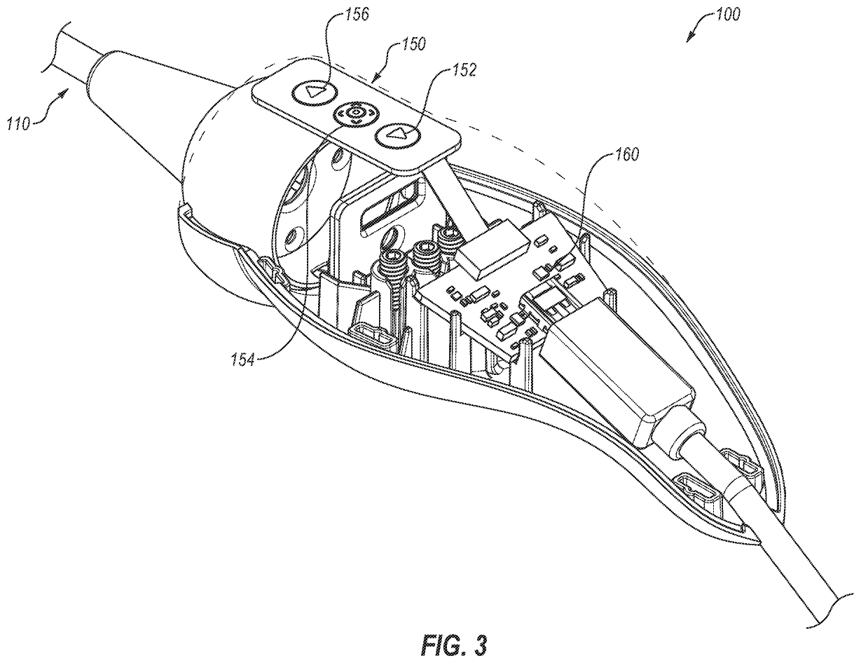

FIG. 3 illustrates the laparoscope 100 of FIG. 2 but with the top portion of the handle shown in phantom to illustrate the internal components of the device. As shown in this drawing, a voltage driver 160 may be provided to facilitate transmission of electrical signals/voltages to the active focus lens 140. Voltage driver 160 may be incorporated into a printed circuit board, which may comprise various other circuits or other electrical components that may be used in the laparoscope 100, none of which are disclosed herein to avoid obscuring the disclosure related to the inventions.

In some embodiments, voltage driver 160 may be configured to generate voltage in steps that may correspond with the number of focus steps that can be used during actuation of active focus lens 140 and therefore the number of focus steps that a user can apply during use of laparoscope 100. In one example of a suitable voltage driver, the voltage driver may be configured, for example, to generate between about 500 and about 2,000 steps of voltage, which may be in increments of, for example about ±50 mV. In some such embodiments, the voltage driver may be configured to generate 1,000 voltage steps for an overall operating voltage range of about ±50 V with individual voltage steps of about ±50 mV.

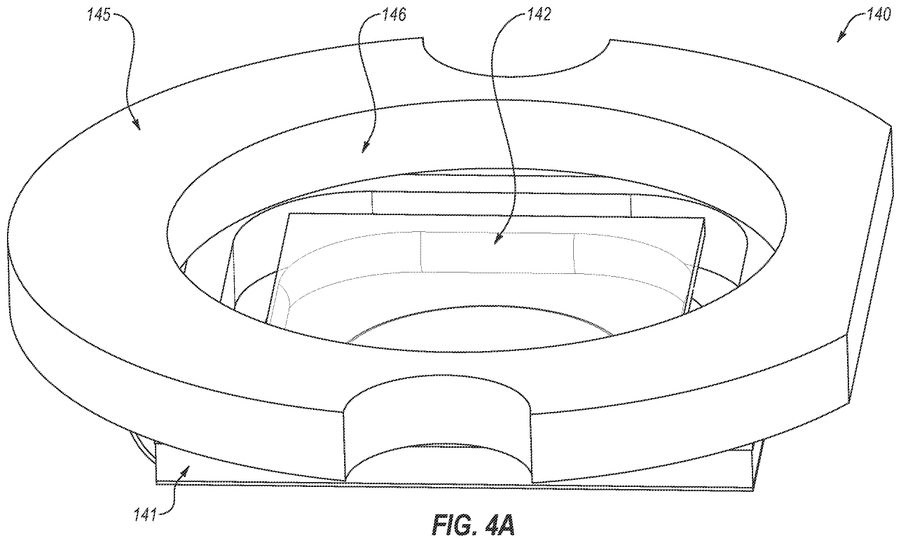

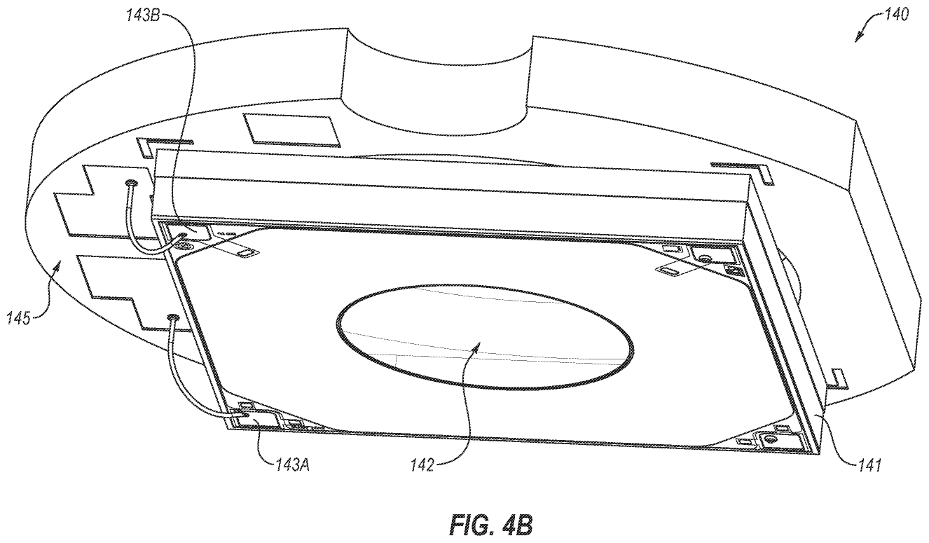

FIGS. 4A and 4B are closeup, perspective views from opposite sides of an active optical lens assembly 140 comprising an active optical lens unit 141 having an active lens 142 and a substrate 145 to facilitate coupling of the active optical lens unit 141 with a laparoscope, endoscope, or other borescope. As shown in these drawings, substrate 145 comprises a central opening 146 that is preferably similar in size and shape to the underlying transparent lens 142, although in some embodiments opening 142 may be larger so prevent blocking light from passing through lens 142.

In preferred embodiments, substrate 145 comprises a printed circuit board, and therefore, as shown in FIG. 4B, may also provide for an electrical coupling between the active optical lens assembly 140 and the actuating/driving elements of the accompanying borescope, such as voltage driver 160 and/or focusing control panel 150, for example. Thus, FIG. 4B further illustrates a lower terminal electrical coupling 143A and an upper terminal electrical coupling 143B, which extend between substrate 145 and respective upper and lower electrodes, which may be used to apply stepped voltages to opposing actuators of the lens assembly 140.

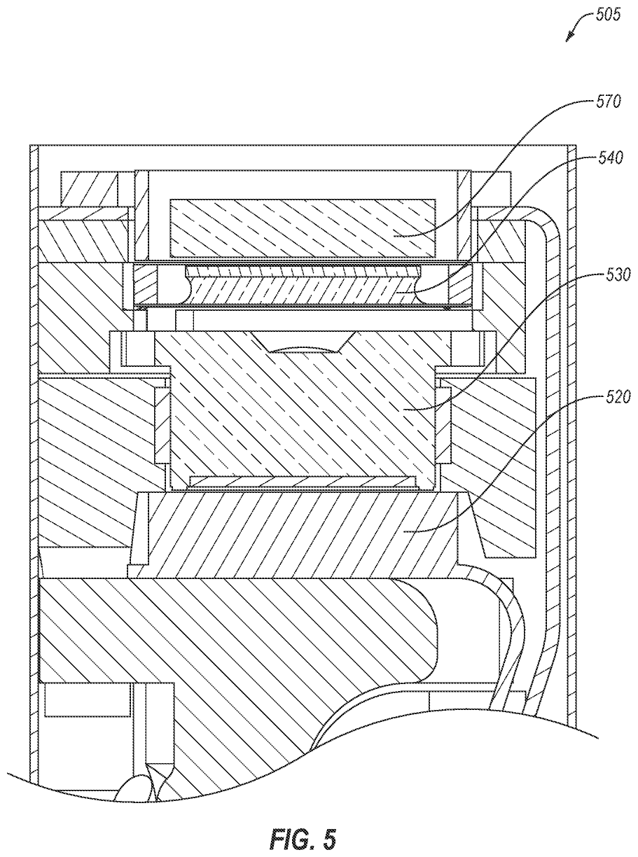

FIG. 5 illustrates a tip assembly 505 of a borescope including active/tunable lens components according to some embodiments. As shown in this figure, tip assembly 505 comprises an image sensor 520, a fixed focus lens 530, and an active/variable focus lens 540, as discussed throughout this disclosure. As described above, in some embodiments, lens 540 may comprise a lens assembly that may include a PCB or other substrate to facilitate coupling of the lens assembly, both electrically and mechanically, to other elements of a laparoscope or other borescope. In addition, tip assembly 505 further comprises a cover 570, which may comprise a glass or other transparent material, at least in part, and may be used to protect the underlying components.

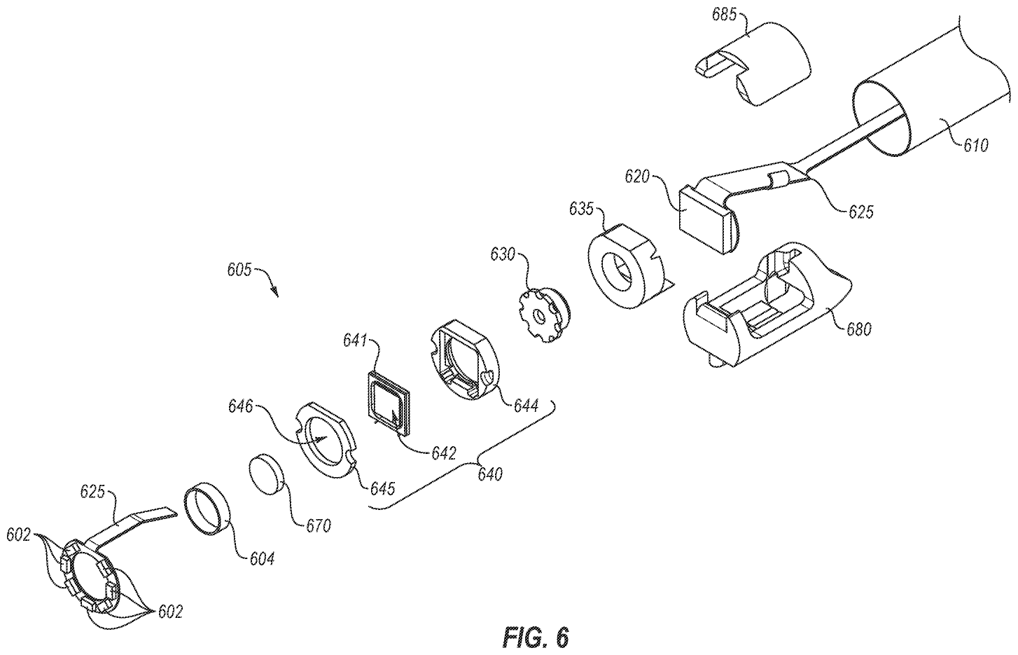

FIG. 6 is an exploded view of a tip assembly 605 of a medical borescope according to other embodiments. As shown in this figure, tip assembly may be positioned at the distal end of a tube 610 or another shaft. Although not shown in this figure, it should be understood that the proximal end of the borescope may comprise a handle, such as, for example, the handle shown in FIGS. 2 and 3.

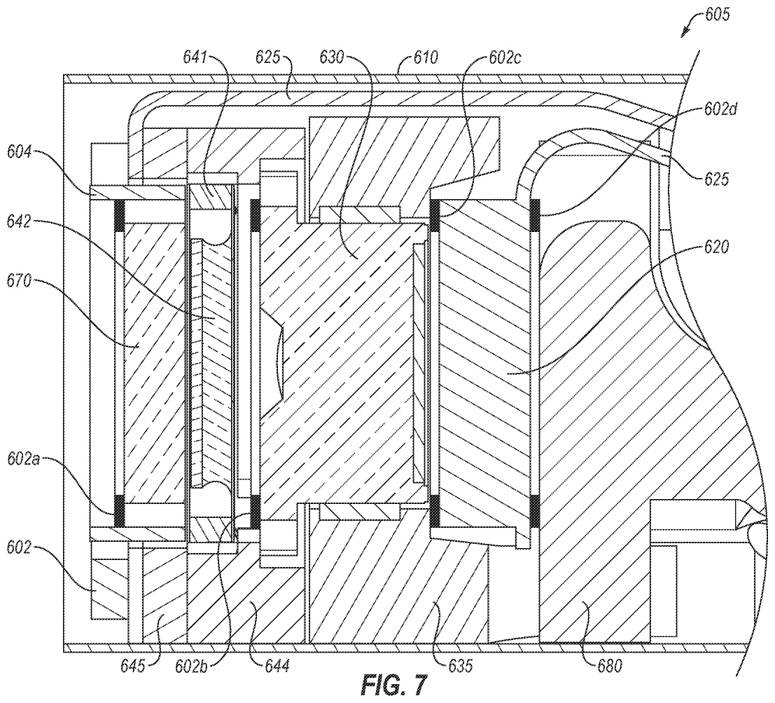

An array of lights, such as LEDs 602, are positioned in a ring formation at the distal end of the tip assembly 605, as shown in both FIG. 6 and in the cross-sectional view of FIG. 7. LEDs 602 may be electrically coupled to a power source and/or other electrical components via, for example, a flexible printed circuit board 625, or another suitable set of electrical wires. A light shield 604 may be provided in order to prevent, or at least inhibit, light from LEDs 602 directly entering the pathway to image sensor 620. Thus, in the depicted embodiment, light shield 604 is positioned in a ring formation with a radius slightly smaller than that of the ring of LEDs 602. Light shield 604 may also protrude slightly beyond the distal extent of LEDs 602, as shown in FIG. 7.

A transparent cover, such as cover glass 670 may be positioned within light shield 604. Again, light shield 604 may be configured to prevent or at least inhibit light from LEDs 602, or another light source, from directly passing therethrough rather than reflecting off of body tissues and then through cover glass 670.

An active/variable focus optical lens assembly 640 may be positioned adjacent to cover glass 670. Active focus optical lens assembly 640 comprises an active optical lens unit 641 having an active lens 642 and a substrate 645 to facilitate coupling of the active optical lens unit 641 within a tube of a scope. As shown in FIGS. 6 and 7, substrate 645 comprises a circular, central opening 646 that is preferably similar in size and shape to the underlying transparent lens 642.

Substrate 645 may comprise a printed circuit board and may thereby provide for both a physical and an electrical coupling between the active optical lens assembly 640 and the actuating/driving elements of the borescope, such as a voltage driver and/or focusing control panel, as previously described. As previously mentioned, active lens 642 may be configured to be actively re-shaped, preferably in real time, to allow for automatic or manual refocusing of images during use. As also previously mentioned, in preferred embodiments, a piezoelectric actuator may be used to accomplish this lens reshaping/refocusing. The piezoelectric actuator may be positioned on adjacent to a thin membrane, such as a glass membrane, which may, in turn deform a polymer, which may be sandwiched between two transparent layers, such as glass layers. The piezoelectric material may be coupled with a tunable voltage source to allow for real-time focusing of the active lens.

As shown in FIG. 6, various elements within tip assembly 605 may have an at least substantially circular shape in cross-section with the exception of one flat side, which may be desirable to provide a more stable coupling within the tube of the scope. For example, substrate 645 and active lens housing 644 may each comprise a flat side.

A fixed lens or camera module 630 may be positioned adjacent to the active lens assembly 640. Fixed lens 630 may be positioned within a fixed lens housing 635, which may also comprise one flat side, as previously mentioned in connection with elements of the active lens assembly 640.

An image sensor 620 may be positioned adjacent to the fixed lens 630, and may be coupled to a flexible PCB 625 that may extend to the handle (not shown) of the scope.

At least a subset of the aforementioned elements of tip assembly 605 may be positioned within a tip assembly housing 680, which may comprise slots or other structures configured to allow for mounting or otherwise coupling of various elements of the assembly. In the depicted embodiment, tip assembly housing 680 may comprise a stable location for mounting fixed lens housing 635 and an internal passage for various elements, such as wires and/or a flexible PCB 625, and in some embodiments articulating links to allow the tip to articulate in one or more directions as dictated by operator control elements. A cap 685 may be provided for tip assembly housing 680, which may allow for access to various elements during assembly.

Tip assembly 605 further comprises various heating elements that may be used to prevent or at least inhibit fogging of the surface or surfaces exposed to the environment at the distal end of the tip. In the depicted embodiment, this surface of concern is cover glass 670. However, it is contemplated that, in alternative embodiments, this outermost/exposed surface may comprise a lens.

Most preferably, a heating element 602a may be positioned on, or adjacent to, cover glass 670 itself. However, other locations for heating elements are possible and may be used in addition to, or as an alternative location to, the depicted location of heating element 602a. For example, a heating element 602b may be positioned in between cover glass 670—or another exterior element exposed to the ambient environment—and one or more of the lens elements. Thus, heating element 602b is shown positioned immediately distal of and adjacent to lens 630. Still other possible locations for heating elements are shown at 602c and 602d. Heating element 602c is positioned in between image sensor 620 and lens 630 and heating element 602d is shown positioned just proximal of image sensor 620. Again, although FIG. 7 depicts each of the aforementioned heating elements 602a-602d, it should be understood that, in some embodiments, just one of these heating elements may be used.

Heating elements may comprise any element capable of generating sufficient heat to avoid, or at least inhibit, condensation on the cover glass or other exterior element of the tip. In some embodiments, suitable heating elements may simply comprise one or more resistors. Such resistors can be driven with a fixed voltage. To optimize power consumption, the resistance can be lowered until the desired amount of heat is generated. For efficiency, it may be preferred to place the resistor(s) on or as close as possible to the cover glass 670 or another transparent element exposed to the ambient environment. Thus, again, the location of heating element 602a may be most desirable for certain applications. However, it is contemplated that sufficient heat may be generated in other locations to have a similar effect if the preferred location is occupied or otherwise not feasible. In preferred embodiments, the heating element is no more than about 2 cm from the glass or other exterior surface. However, in other embodiments, the heating element(s) may be up to about 10 cm from this location.

Although heating elements 602c and 602d are shown positioned adjacent to the image sensor 620, because image sensors are often adversely sensitive to heat, it should be understood that insulative elements may be positioned in between the image sensor 620 and the heating element(s), or the heating element(s) may be positioned spaced apart from the image sensor 620 in other locations within the tip assembly 605 if desired.

As those of ordinary skill in the art will appreciate, in the case of “chip-on-tip” scopes, such as the embodiment depicted in FIG. 7, in which LEDs are integrated into the tip, heating elements may be unnecessary due to the heat created by the LEDs themselves. Thus, it is contemplated that the heating provided by these elements may be more suitable to use in connection with scopes having light pipes or fibers, for example, or where there is an insufficient number of LEDs or an insufficient amount of heat generated thereby. Thus, whereas an LED 602 is shown in the embodiment of FIG. 7, it should be understood that heating elements may instead be incorporated into tips/scopes that lack LEDs, or that contain fewer LEDs that are included in the ring array of LEDs shown in FIG. 6.

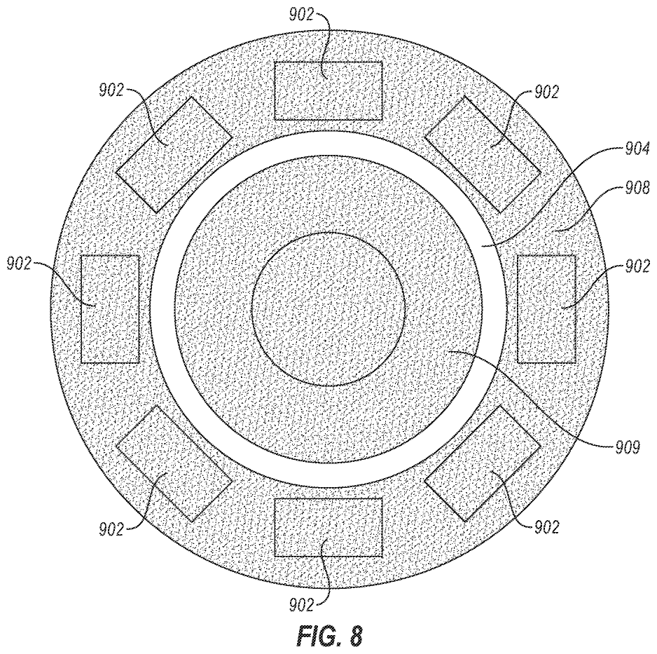

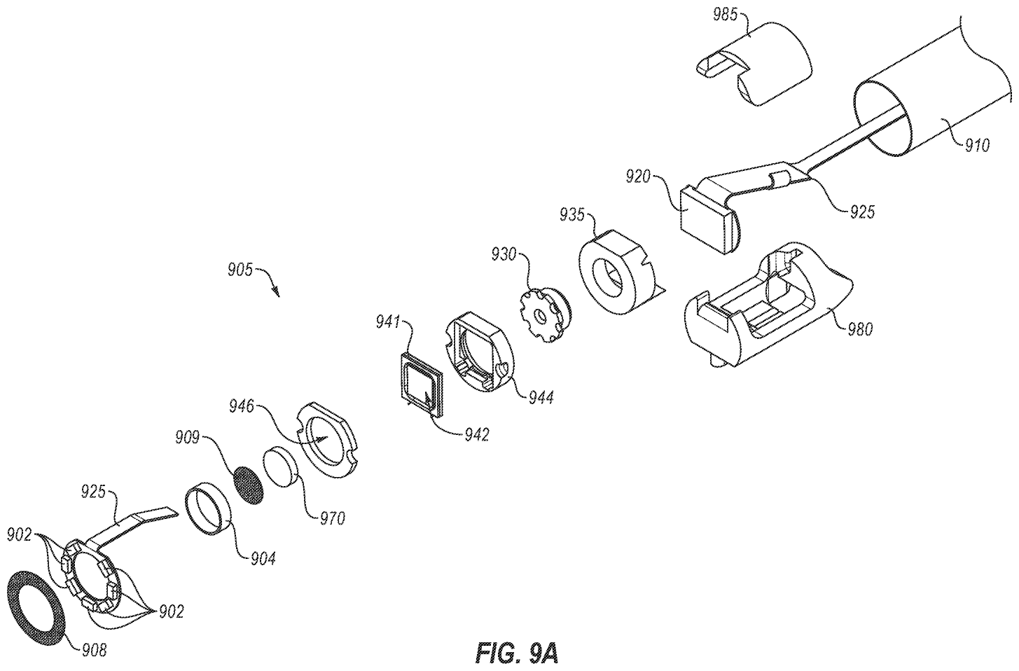

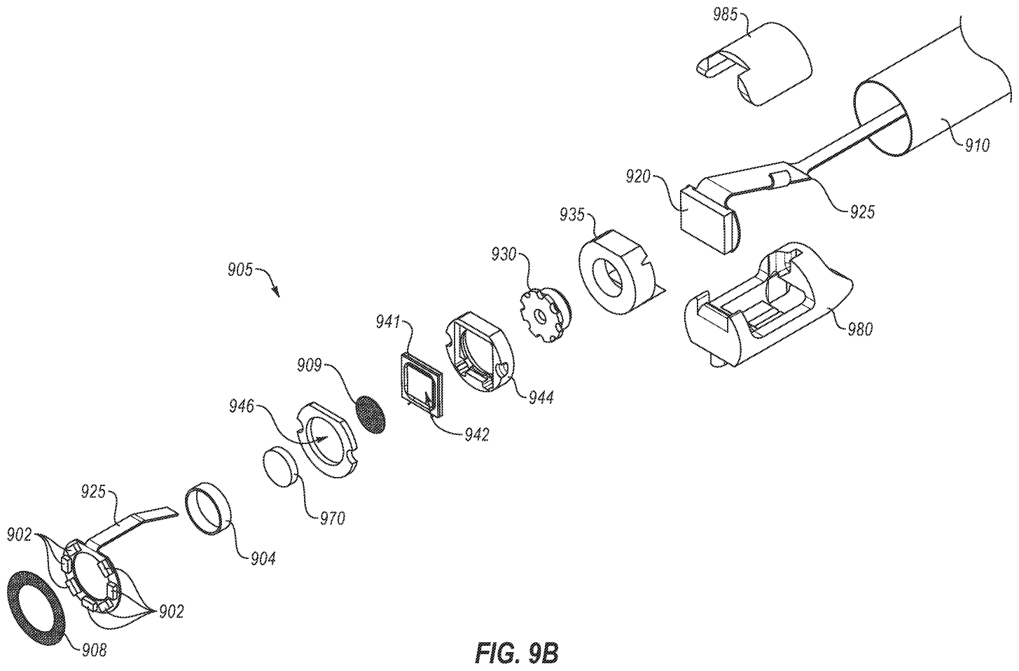

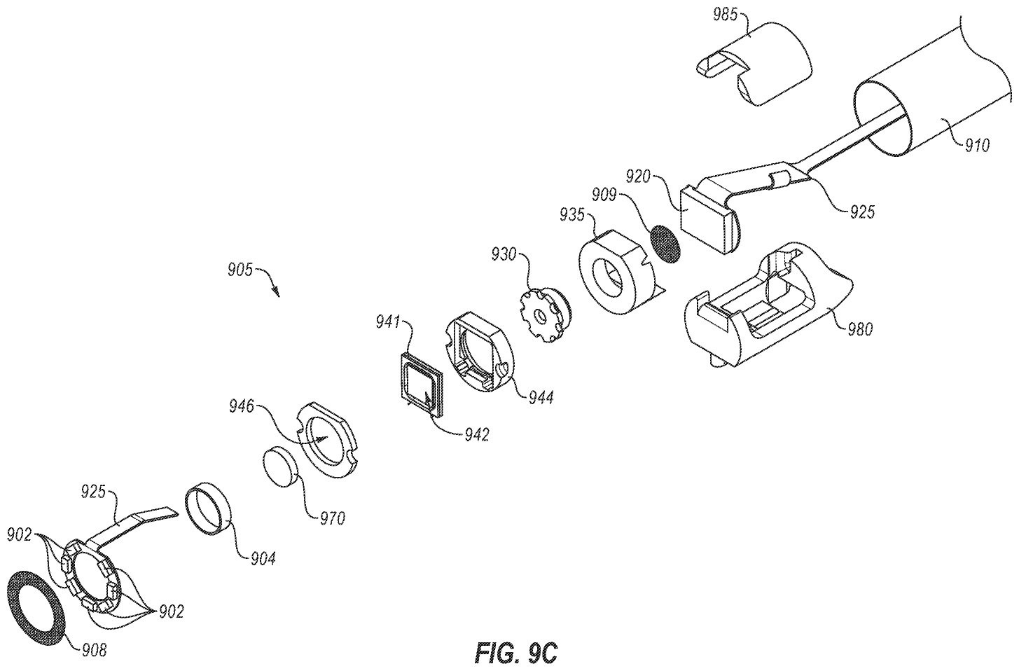

FIG. 8 depicts another alternative embodiments of a medical borescope configured to reduced specular glare/specular highlights often caused by wet or moist tissues or other surfaces being illuminated and imaged. Specular highlights when visualizing internal surfaces during surgery can be distracting or even dangerous, as they may impair diagnostics. Specular highlights are often caused by direct light in the direct single-reflection path from the light source to the lens input. The direct, single-path light largely remains the same polarization as the light source, whereas light from multiple reflection paths tends to become a random polarization. Thus, the embodiment of FIG. 8, and of FIGS. 9A and 9B, are configured to reduce specular highlights by removing or at least decreasing the intensity of the direct, single-reflection light.

In the depicted embodiments, this is accomplished by use of polarizing films that are rotated with respect to one another. More particularly, a first polarizer, such as a first polarizing film, 908 is positioned in the path of the light source, which in this case comprises an array of LEDs 902, but may comprise alternative light sources in other embodiments. Because the array of LEDs 902 is formed in the shape of a ring, the film 908 is also ring-shaped so as to ensure that all of the light being transmitted from the LEDs 902 is polarized. However, it should be understood that if the light source is positioned closer to the center of the tube/shaft of the scope, the first polarizing film 908 may be a circular, rectangular, or other non-ring-shaped polarizing film, so long as it covers the LED(s) or other light emitting source within the scope.

A second polarizer, such as a second polarizing film 909, which is rotated with respect to the first polarizer/polarizing film 908, may be positioned somewhere within the path of the reflected light that is entering one or more of the lenses and/or the image sensor. Preferably, polarizing film 909 is rotated by 90 degrees with respect to polarizing film 908, so as to minimize the specular highlights that can be distracting to practitioners during use of the scope. As shown in FIG. 8, due once again to the outer ring shape of the LEDs 902, polarizing film 909 may comprise a simple circular shape that is positioned within the ring-shaped polarizing film 908. A light shield 904 may be positioned in between the outer ring of LEDs 902 and corresponding ring-shaped polarizing film 908 and the reflection light path within which polarizing film 909 is placed so as to prevent light from LEDs 902 from directly entering this path.

Because use of polarizing films or other polarizers may reduce the intensity of the light, in embodiments comprising such polarizers, it may be desirable to compensate for this reduction in light intensity by increasing the intensity of LEDs 902 or another light source, and/or by increasing the gain in image processing. Some embodiments may therefore be configured to allow a user to increase light gain and/or intensity at will, such as in light-starved scenes (such as when imaged objects are relatively far away).

FIGS. 9A-9C are exploded views depicting various alternative embodiments comprising polarizers that illustrate exemplary locations for the aforementioned polarizing screens. In the embodiment of FIG. 9A, a ring-shaped polarizing film 908 is positioned adjacent to the ring of LEDs 902 at the distal end of the tip assembly 905. Another polarizing film 909, which is rotated, preferably by 90 degrees or about 90 degrees, with respect to the first polarizing film 908, is positioned adjacent to the cover glass 970. This ensures that that light reflected from the LEDs 902, which passes through the first polarizing film 908, passes through the second polarizing film 909 prior to entering the various lenses, such as variable focus lens 942 and fixed lens 930, and ultimately, image sensor 920 within the tube 910 of the scope. Again, because the pathway for incoming/reflected light is within the pathway for outgoing light from LEDs 902, polarizing film 909 is circular and has a diameter that is less than or equal to the inner diameter of the ring of polarizing film 908. Of course, a wide variety of alternative shapes and configurations will be apparent to those of ordinary skill in the art after having received the benefit of this disclosure.

Tip assembly 905 may otherwise be identical or similar to tip assemblies of embodiments previously discussed. Thus, tip assembly 905 may further comprise a light shield 904 to prevent, or at least inhibit, light from LEDs 902 directly entering the pathway to image sensor 920. Tip assembly 905 may further comprise an active/variable focus optical lens assembly, which may itself comprise an active optical lens unit 941 having an active lens 942 and a substrate 945 having a circular opening 946, all of which may be configured to be positioned within a housing 944.

Substrate 945 may, once again, comprise a printed circuit board and may thereby provide for both a physical and an electrical coupling between the active optical lens assembly and the actuating/driving elements of the borescope, such as a voltage driver and/or focusing control panel. As also previously mentioned, active lens 942 may be configured to be actively re-shaped, preferably in real time, to allow for automatic or manual refocusing of images during use, which may be done by way of, for example, a piezoelectric actuator.

A fixed lens or camera module 930 may be positioned adjacent to the active lens assembly, which, again, may comprise active optical lens unit 941, active lens 942, substrate 945, and housing 944. Fixed lens 930 may be positioned within a fixed lens housing 935. Image sensor 920 may be positioned adjacent to the fixed lens 930, and may be coupled to a flexible printed circuit board 925 that may extend to the handle (not shown) of the scope.

At least a subset of the aforementioned elements of tip assembly 905 may be positioned within a tip assembly housing 980, which may comprise slots or other structures configured to allow for mounting or otherwise coupling of various elements of the assembly, along with, in some embodiments, a cap 985 to allow for access to, and/or enclosure of, various elements of the assembly.

FIG. 9B illustrates an alternative embodiment of a tip assembly 905 having the same components as the tip assembly of FIG. 9A, but with the interior/reflected light path polarizing film 909 positioned at a different location. In particular, polarizing film 909 is positioned adjacent to the active lens assembly of the housing within the scope rather than adjacent to the exterior cover glass 970. It is contemplated that polarizing film 909 of the embodiment of FIG. 9B may therefore, in some embodiments, be part of an assembly of elements making up the active lens.

Yet another example of an alternative location for polarizing film 909 is shown in FIG. 9C, which includes all of the same basic elements as previously shown in FIGS. 9A and 9B, but illustrates the second polarizing film 909 within the reflected light path as being adjacent to the image sensor 920. As those of ordinary skill in the art will appreciate, the second polarizing film 909 may therefore be positioned at any location in between the element exposed to the ambient environment, which in the case of the embodiments of FIGS. 9A-9C is cover glass 970, and the image sensor 920.

Although the articulating mechanisms are not described in detail herein, in preferred embodiments, at least a portion of the shaft/tube of the scope may be configured to articulate in one or more directions. A flexible PCB (printed circuit board) may be used to run the wires between various elements from the tip to the handle. The present inventors have discovered that using a flexible PCB that has a natural bend and/or is intended to bend in only one direction may result in problems when used in connection with an articulating scope. For example, the PCB and/or wires contained therein may be prone to breaking or other damage when articulating against the natural bending direction of the PCB.

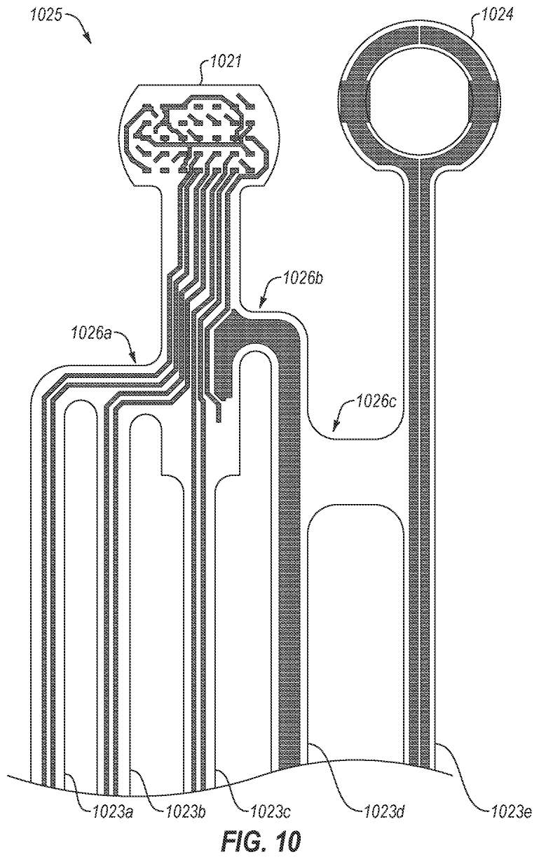

As a result, it may be desirable to reconfigure a typical PCB such that the electrical wiring extends within a series of individual strips rather than a single strip and fold them together, preferably in an alternating folding pattern or “accordion-style” fold. FIG. 10 illustrates an embodiment of a flexible PCB 1025 according to some such embodiments. As shown in this figure, flexible PCB 1025 comprises five strips of wires, some of which may be interconnected physically and/or electrically. In particular, rather than providing all of the wires shown along a single strip, strips 1023a, 1023b, 1023c, 1023d, and 1023e are provided. Each of flexible PCB strips 1023a-1023d is electrically coupled with an image sensor PCB pad 1021, which, as discussed in greater detail below, is configured to be electrically and/or physically coupled with an image sensor of the scope. The fifth strip 1023e of the flexible PCB strip assembly 1025 comprises a ring/LED PCB pad 1024, which allows wiring to be coupled to each of the LEDs, such as LEDs 902, to be electrically coupled to the flexible PCB assembly 1025. As also shown in FIG. 10, interconnecting pieces may be positioned between adjacent strips such that each of the strips of the assembly is physically connected as a unit.

In some embodiments, the combined widths of each of the strips may be greater than the inner diameter of the tube of the scope. Thus, if the tube has an inner diameter of about 3.5 mm, for example, in some embodiments, rather than providing a single flexible PCB strip that is 3.5 mm or less in width, each strip may be about 1 mm wide, which results in a total width of about 5 mm.

Preferably, the interconnecting pieces are configured so as to allow for folding the assembly together as a unit. Thus, in some embodiments, the interconnecting pieces may provide spacing between each adjacent strip of at least about the width of an individual strip. For example, if each strip is about 1 mm wide, as mentioned above, the spacing between each adjacent strip may be at least about 1 mm. Additional spacing may be provided in between some of the adjacent strips, such as between strips 1023d and 1023e. Thus, in some embodiments, the spacing between adjacent strips may be between about 1-3 times the width of a single individual strip, or between about 1 mm and about 3 mm in the case of a flexible PCB assembly for a tube having an inner diameter of about 3.5 mm, for example.

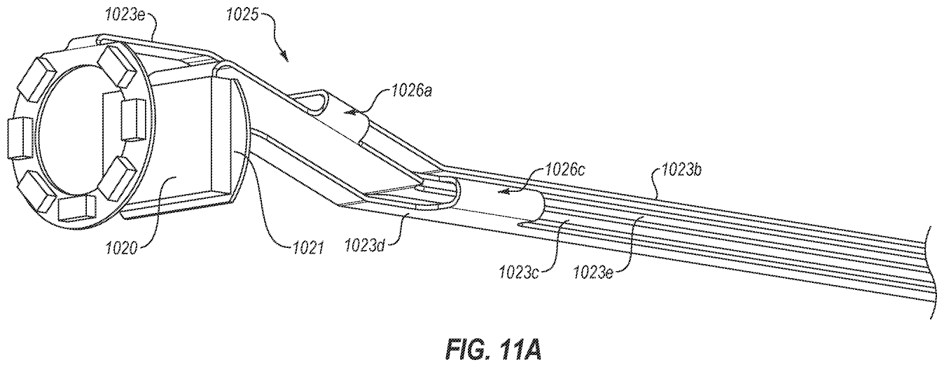

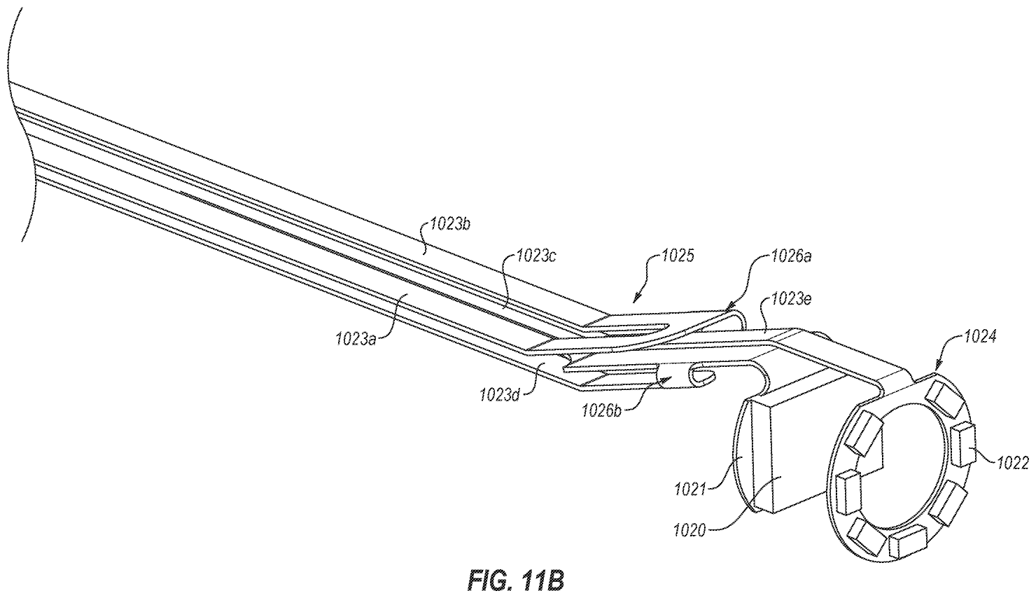

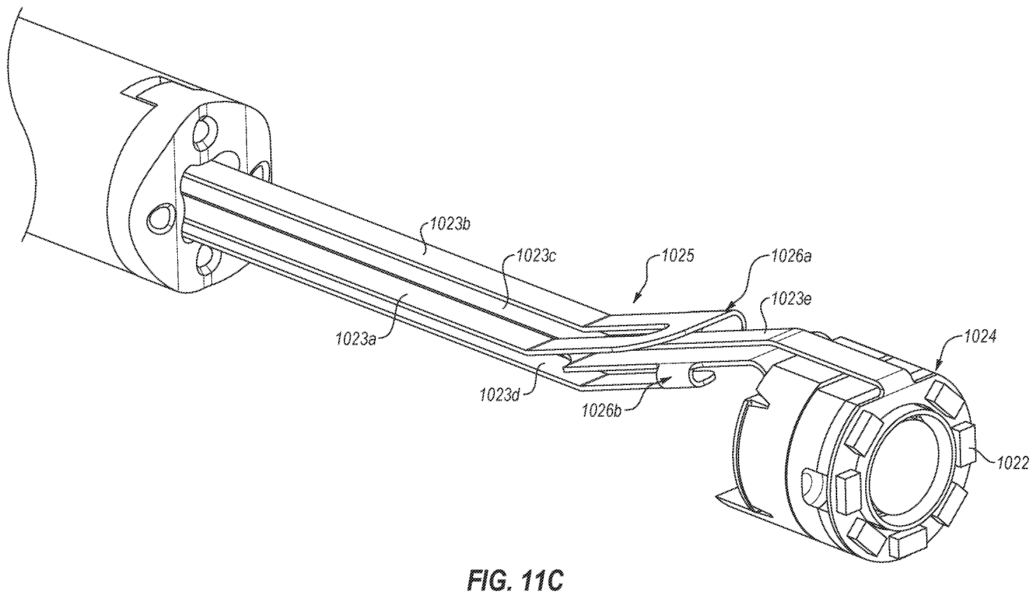

As previously mentioned, to avoid tearing or other damage to the wires, the individual strips 1023a-1023e of assembly 1025 may be folded together, preferably in an alternating/accordion-style manner, within the tube of the scope. Thus, FIGS. 11A-11C depict various views of assembly 1025 in a folded configuration with particular elements of a tip assembly of a scope coupled thereto. As shown in these figures, there are three major folds going in opposite directions, namely, a first fold 1026a extending in a first direction, a second fold 1026b extending in a second direction opposite from the first direction, and a third fold 1026c extending in the direction of the first fold 1026a. The locations of these three folds can also be seen in the unfolded assembly 1025 shown in FIG. 10.

FIGS. 11A-11C further illustrate the relative locations of certain components of a tip assembly along with the flexible, partitioned, PCB assembly 1025, including image sensor 1020, which has image sensor PCB pad 1021 coupled thereto, along with LEDs 1022, which has ring/LED PCB pad 1024 coupled thereto. FIG. 11C further illustrates how the aforementioned folding may take place proximally of the full stack of lens elements of the tip assembly, which may include both active and fixed lenses and associated actuation elements, a light shield, and/or other elements, including but not limited to those previously mentioned. Although it is contemplated that, in preferred embodiments, partitioned, flexible PCB assembly 1025 may extend within a tube of the scope, it is also contemplated that, in alternative embodiment, one or more strips, or possibly the entire assembly, could be positioned on an exterior surface of the tube/shaft of the scope, possibly within an exterior sheath or the like.

It will be understood by those having skill in the art that changes may be made to the details of the above-described embodiments without departing from the underlying principles presented herein. Any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein. The scope of the present invention should, therefore, be determined only by the following claims.

1. A medical borescope, comprising:
a handle;
a tube coupled to the handle; and
a tip assembly positioned at or adjacent to a distal tip of the tube, wherein the tip assembly comprises:
a light source;
an image sensor;
a fixed focus lens; and
a variable focus lens configured to allow an operator of the medical borescope to adjust a focal distance of the medical borescope during use.

2. The medical borescope of claim 1, wherein the variable focus lens comprises a variable focus lens assembly comprising a substrate and an active optical lens unit configured to be reshaped during use to change the focal distance of the medical borescope.

3. The medical borescope of claim 2, wherein the substrate comprises a printed circuit board.

4. The medical borescope of claim 3, wherein the substrate comprises a central opening configured to allow light to pass therethrough and reach the active optical lens unit.

5. The medical borescope of claim 3, wherein the printed circuit board is configured to provide an electrical interface with the fixed focus lens.

6. The medical borescope of claim 2, wherein the substrate comprises a ring shape having a flat side configured to allow electrical wires to pass by the substrate within the tube.

7. The medical borescope of claim 1, further comprising a tunable voltage source configured to allow for selective adjustment of the focal distance by changing a shape of the variable focus lens.

8. The medical borescope of claim 7, wherein the tunable voltage source comprises a voltage driver.

9. The medical borescope of claim 1, further comprising a piezoelectric actuator configured to change the focal distance by changing a shape of the variable focus lens.

10. The medical borescope of claim 1, wherein the medical borescope is configured to provide for automatic focusing.

11. The medical borescope of claim 1, further comprising:
a transparent cover positioned at or adjacent to a distal end of the tip assembly; and
a heating element positioned and configured to reduce condensation on the transparent cover during use.

12. A medical borescope, comprising:
a handle;
a shaft coupled to the handle, wherein at least a portion of the shaft is configured to articulate;
an array of LEDs positioned at a tip of the shaft;
a variable focus lens configured to allow for adjustment of a focal distance of the medical borescope during use; and
a flexible printed circuit board extending from the array of LEDs to the handle, wherein the flexible printed circuit board comprises a plurality of flexible strips folded to facilitate bending of the flexible printed circuit board in at least two directions.

13. The medical borescope of claim 12, further comprising an image sensor, wherein the flexible printed circuit board is coupled with the image sensor.

14. The medical borescope of claim 12, wherein the shaft comprises a tube, and wherein the flexible printed circuit board extends through the tube from the array of LEDs to the handle.

15. The medical borescope of claim 14, wherein each of the plurality of flexible strips is folded within the tube.

16. The medical borescope of claim 15, wherein each of the plurality of flexible strips is folded accordion style within the tube.

17. The medical borescope of claim 12, further comprising:
an image sensor;
a first polarized film configured to polarize light from the array of LEDs; and
a second polarized film configured to polarize light entering the image sensor, wherein the first polarized film is rotated with respect to the second polarized film.

18. A medical borescope, comprising:
a handle;
a tube coupled to the handle;
a fixed focus lens; and
a variable focus lens assembly comprising:
a printed circuit board substrate; and
an active optical lens unit configured to be reshaped during use to change the focal distance of the medical borescope.

19. The medical borescope of claim 18, wherein the medical borescope is configured to provide for either automated or manual refocusing of the variable focus lens assembly.

20. The medical borescope of claim 18, wherein the variable focus lens assembly further comprises a piezoelectric actuator.
